(19) United States
(12) Patent Application Publication
(10) Pub. No.: US 2015/0185196 A1
(43) Pub. Date: Jul. 2, 2015

(54) CHARACTERIZATION OF FIELD SITES FOR UTILITY IN AGRONOMIC STRESS TRIALS

(71) Applicant: DOW AGROSCIENCES LLC, Indianapolis, IN (US)

(72) Inventors: Tristan E. Coram, Zionsville, IN (US); Terry R. Wright, Carmel, IN (US); Sachidananda Mishra, Indianapolis, IN (US); Paolo P. Castiglioni, Davis, CA (US)

(21) Appl. No.: 14/567,709

(22) Filed: Dec. 11, 2014

Related U.S. Application Data
(60) Provisional application No. 61/921,268, filed on Dec. 27, 2013.
(60) Provisional application No. 62/065,199, filed on Oct. 17, 2014.

(51) Int. Cl. G01N 33/24

(52) U.S. Cl. CPC G01N 33/24

(57) ABSTRACT

Methods are disclosed for characterizing variability at field sites and for selecting “zones of uniformity” at field sites with little or no variability to enhance the probability of successful agronomic stress trials.

### CROSS-REFERENCE TO RELATED APPLICATIONS

This application claims priority to U.S. Provisional Patent Application Ser. No. 61/921,268, filed Dec. 27, 2014, and U.S. Provisional Patent Application Ser. No. 62/065,199, filed Oct. 17, 2014, the disclosures of which are hereby expressly incorporated by reference herein in their entirety.

### FIELD

The present invention relates to agronomic stress trials and, in particular, to methods for characterizing and selecting field sites for agronomic stress trials.

### BACKGROUND AND SUMMARY

The site selected for planting an agricultural crop may impact agronomic performance of the crop. In particular, variability in physical and/or chemical characteristics of the soil at the site may impact agronomic performance of the crop. For example, if the soil in Plot A differs from the soil in Plot B, the crops planted in Plot A may perform better (e.g., produce a higher yield) than the crops planted in Plot B.

To minimize variability at the site, physical and/or chemical soil data may be collected, analyzed, and used to develop different treatments across the site. Returning to the example above, if the soil data indicates that the soil in Plot A contains more nutrients than the soil in Plot B, extra fertilizer may be applied to the soil in Plot B to minimize variability between Plot A and Plot B. Also, if the soil data indicates that the soil in Plot A retains more moisture than the soil in Plot B, extra water may be applied to the soil in Plot B to minimize variability between Plot A and Plot B. Large-scale soil data is available from the United States Department of Agriculture (USDA) Natural Resource Conservation Service (NRCS) Soil Survey. Small-scale soil data may be determined using the Soil Information System™ (SIS) provided by C3 Consulting, LLC of Fresno, Calif., for example.

Agronomic stress trials are performed to assess agronomic performance of crops under stressed growing conditions, such as water deficit conditions (e.g., limited or no irrigation) or nutrient deficit conditions (e.g., limited or no fertilizer). Soil variability at the test site may impact the outcome of an otherwise controlled stress trial. However, soil data that is available for normal growing conditions may not be applicable to a stress trial involving stressed growing conditions, because crops may respond differently under stressed growing conditions compared to normal growing conditions. Also, soil treatments that are designed to improve soil quality and soil consistency (e.g., fertilizer applications) in normal growing conditions may not be appropriate for a field trial that requires stressed growing conditions.

The present disclosure provides methods for characterizing variability at field sites and for selecting “zones of uniformity” at field sites with little or no variability to enhance the probability of successful agronomic stress trials to generate accurate and reliable phenotyping.

In an exemplary embodiment of the present disclosure, a method is provided for performing an agronomic test at a field site. The method includes: identifying a zone of the field site having minimal variation in at least one predetermined soil parameter, the at least one predetermined soil parameter affecting agronomic performance during the test; planting a crop in the zone of the field site; and subjecting the planted crop to the test.

In another exemplary embodiment of the present disclosure, a method is provided for selecting a field site for an agronomic test. The method includes: planting a test crop; subjecting the planted test crop to the test; determining at least one soil parameter that affects agronomic performance of the test crop during the test; and selecting a zone of the field site having minimal variation in the at least one soil parameter.

In yet another exemplary embodiment of the present disclosure, a method is provided for selecting a field site for an agronomic test. The method includes: planting a first test crop; subjecting the first planted test crop to the test; determining at least one soil parameter that affects agronomic performance of the first test crop during the test; selecting a zone of the field site having minimal variation in the at least one soil parameter; planting a second test crop in the zone; and subjecting the second planted test crop to the test. In certain embodiments, the second test crop is planted remotely from the first test crop.

The above mentioned and other features of the invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIG. 1 illustrates an exemplary method of the present disclosure for characterizing a field site and selecting a “zone of uniformity” at the field site for an agronomic stress trial;

FIG. 2 is a plan view of an exemplary field site having a “zone of uniformity”;

FIG. 3 is a schematic elevational view of a system for collecting data at the field site;

FIG. 4 illustrates an exemplary computer for use in the method of FIG. 1;

FIGS. 5A and 5B illustrate exemplary uniformity maps, where FIG. 5A depicts a “zone of uniformity” and FIG. 5B lacks a “zone of uniformity”;

FIG. 6 illustrates another exemplary method of the present disclosure for characterizing a field site and selecting a “zone of uniformity” at the field site for an agronomic stress trial;

FIGS. 7A-7C illustrate exemplary uniformity maps associated with the Example; and

FIG. 8 illustrates an exemplary uniformity map associated with the Example and identifying two “zones of uniformity.”

### DETAILED DESCRIPTION OF THE DRAWINGS

The embodiments disclosed below are not intended to be exhaustive or to limit the invention to the precise forms disclosed in the following detailed description. Rather, the embodiments are chosen and described so that others skilled in the art may utilize their teachings.

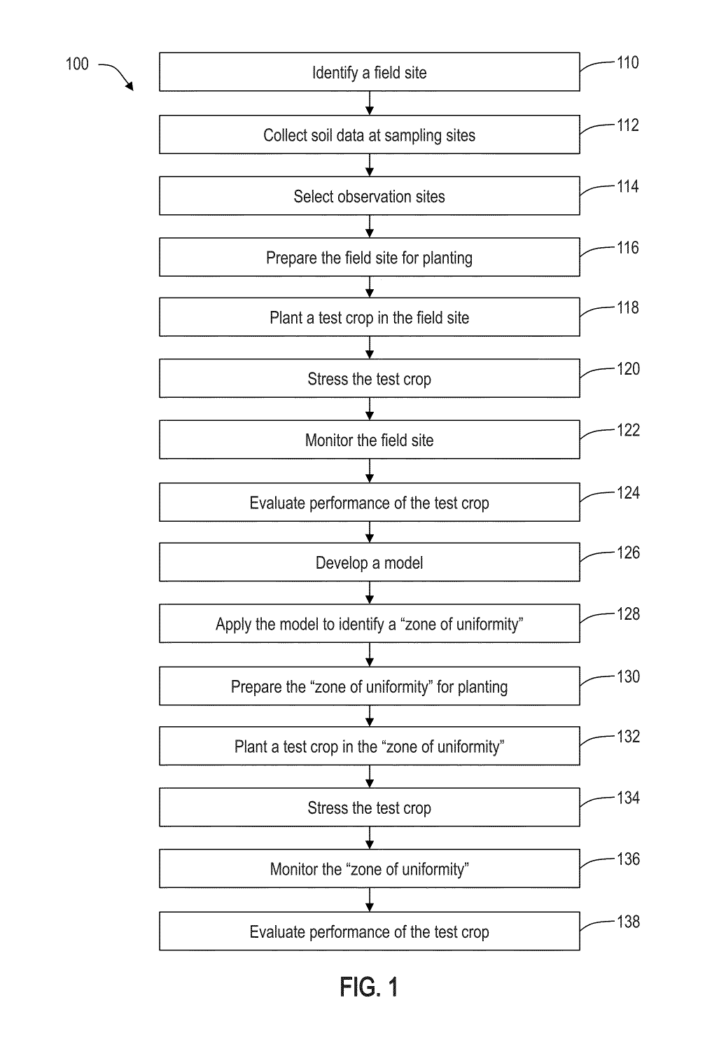

Referring initially to FIG. 1, an exemplary method 100 is provided for characterizing a field site and selecting a “zone of uniformity” at the field site for an agronomic stress trial. The following method 100 may be used to perform the agronomic stress trial for a particular crop and for a particular stress condition.

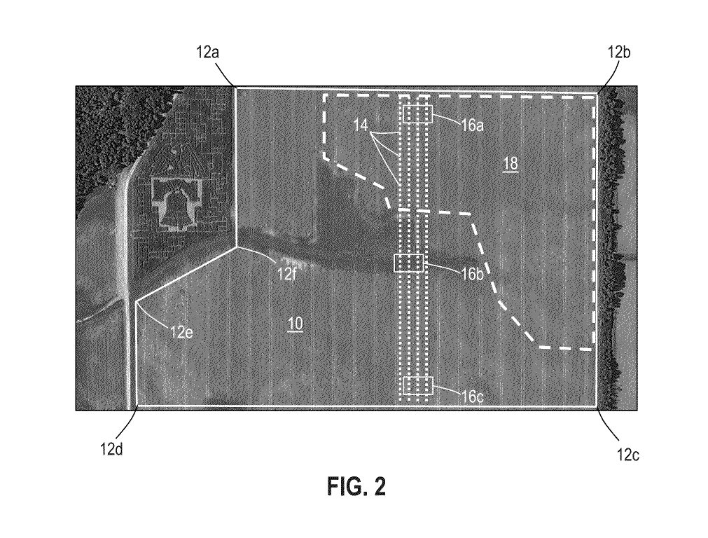

In step 110 of method 100, a field site 10 is identified. An exemplary field site 10 is shown with solid borders in FIG. 2. The field site 10 may be defined by the geographic coordinates of each corner or border, for example, or by another suitable method. In the illustrated embodiment of FIG. 2, the field site 10 is defined by the geographic coordinates of corners 12a-12f. The size of the field site 10 may vary. For example, the size of the field site 10 may be about 20, 40, 60, 80 or 100 acres or more. The shape of the field site 10 may also vary. Although the illustrative field site 10 of FIG. 2 is hexagonal in shape, the field site 10 may also be circular, triangular, rectangular, or irregular in shape, for example.

Returning to FIG. 1, in step 112 of method 100, soil data is collected throughout the field site 10 to evaluate various physical and/or chemical soil parameters. The collecting step 112 may occur before a planting step 118 and a stressing step 120, which are described further below. Exemplary physical soil parameters (P1-P19) for the collecting step 112 are presented in Table 1 below, and exemplary chemical soil parameters (C1-C47) for the collecting step 112 are presented in Table 2 below.

TABLE 1
Physical Soil Parameters

| Number | Parameter | Units |
|---|---|---|
| P1 | Bulk Density (at five levels) | grams/cubic centimeter |
| P2 | Surface Clay | % |
| P3 | Sub-surface Clay | % |
| P4 | Depth to Root Restriction | inches @ psi |
| P5 | Drainage Potential | dimensionless index |
| P6 | Plant Available Water | Inches |
| P7 | Root Zone Field Capacity | Inches |
| P8 | Root Zone Permanent Wilting Point | Inches |
| P9 | Root Zone Plant Available Water | Inches |
| P10 | Root Zone Saturated Hydraulic | inches/hour |
| | Conductivity | |
| P11 | Root Zone Saturation | Inches |
| P12 | Surface Sand | % |
| P13 | Sub-surface Sand | % |
| P14 | Surface Texture | USDA Texture |
| | | Classification |
| P15 | Sub-surface Texture | USDA Texture |
| | | Classification |
| P16 | Surface Horizon Thickness | Inches |
| P17 | Sub-surface Horizon Thickness | Inches |
| P18 | Surface Compaction | Psi |
| P19 | Sub-surface Compaction | Psi |

TABLE 2
Chemical Soil Parameters

| Number | Parameter | Units |
|---|---|---|
| C1 | Surface Ammonium | Ppm |
| C2 | Sub-surface Ammonium | Ppm |
| C3 | Surface Boron | Ppm |
| C4 | Sub-surface Boron | Ppm |
| C5 | Surface Calcium | Ppm |
| C6 | Sub-surface Calcium | Ppm |
| C7 | Surface Calcium Base Saturation | % |
| C8 | Sub-surface Calcium Base Saturation | % |
| C9 | Surface Calcium Magnesium Ratio | Ratio |
| C10 | Sub-surface Calcium Magnesium Ratio | Ratio |
| C11 | Surface Cation Exchange Capacity | meq/100 g |
| C12 | Sub-surface Cation Exchange Capacity | meq/100 g |
| C13 | Surface Copper | Ppm |
| C14 | Sub-surface Copper | Ppm |
| C15 | Surface Iron | Ppm |
| C16 | Sub-surface Iron | Ppm |
| C17 | Surface Magnesium | Ppm |
| C18 | Sub-surface Magnesium | Ppm |
| C19 | Surface Magnesium Base Saturation | % |
| C20 | Sub-surface Magnesium Base Saturation | % |
| C21 | Surface Manganese | Ppm |
| C22 | Sub-surface Manganese | Ppm |
| C23 | Surface Nitrate-N | Ppm |
| C24 | Sub-surface Nitrate-N | Ppm |
| C25 | Nutrient Holding Capacity | dimensionless index |
| C26 | Surface Organic Matter | % |
| C27 | Sub-surface Organic Matter | % |
| C28 | Surface pH | pH units |
| C29 | Sub-surface pH | pH units |
| C30 | Surface Phosphorus | Ppm |
| C31 | Sub-surface Phosphorus | Ppm |
| C32 | Surface Phosphorus Availability | dimensionless index |
| C33 | Sub-surface Phosphorus Availability | dimensionless index |
| C34 | Surface Potassium | Ppm |
| C35 | Sub-surface Potassium | Ppm |
| C36 | Surface Potassium Base Saturation | % |
| C37 | Sub-surface Potassium Base Saturation | % |
| C38 | Surface Potassium Magnesium Ratio | Ratio |
| C39 | Sub-surface Potassium Magnesium Ratio | Ratio |
| C40 | Surface Sodium | Ppm |
| C41 | Sub-surface Sodium | Ppm |
| C42 | Surface Sodium Base Saturation | % |
| C43 | Sub-surface Sodium Base Saturation | % |
| C44 | Surface Soluble salt | dS/m |
| C45 | Sub-surface Soluble salt | dS/m |
| C46 | Surface Zinc | Ppm |
| C47 | Sub-surface Zinc | Ppm |

During the collecting step 112, the soil data may be collected at a plurality of surface and sub-surface sampling sites 14 located across the field site 10, as shown in FIG. 2. For purposes of illustration, 4 rows of sampling sites 14 are shown in FIG. 2, but additional sampling sites 14 may be provided across the field site 10. The number, density, and pattern of the sampling sites 14 may vary. For example, in certain embodiments, the sampling sites 14 may be arranged in a grid-shaped pattern that covers nearly the entire surface of the field site 10. The soil parameters evaluated at each sampling site 14 may also vary.

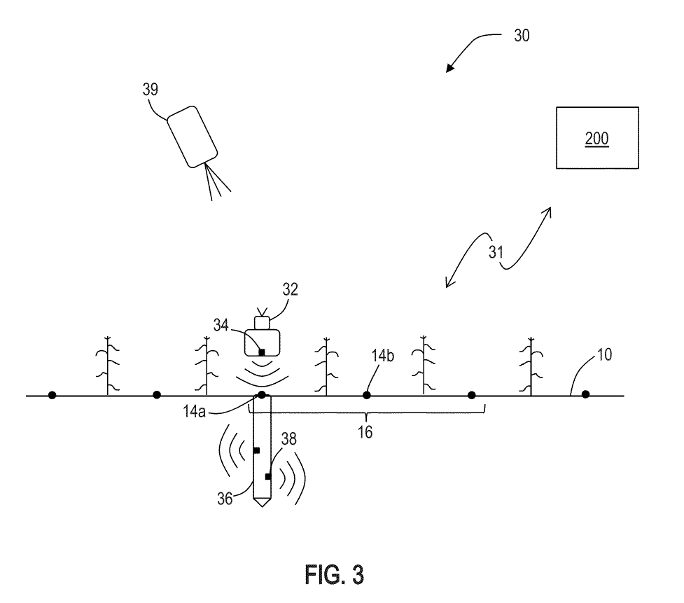

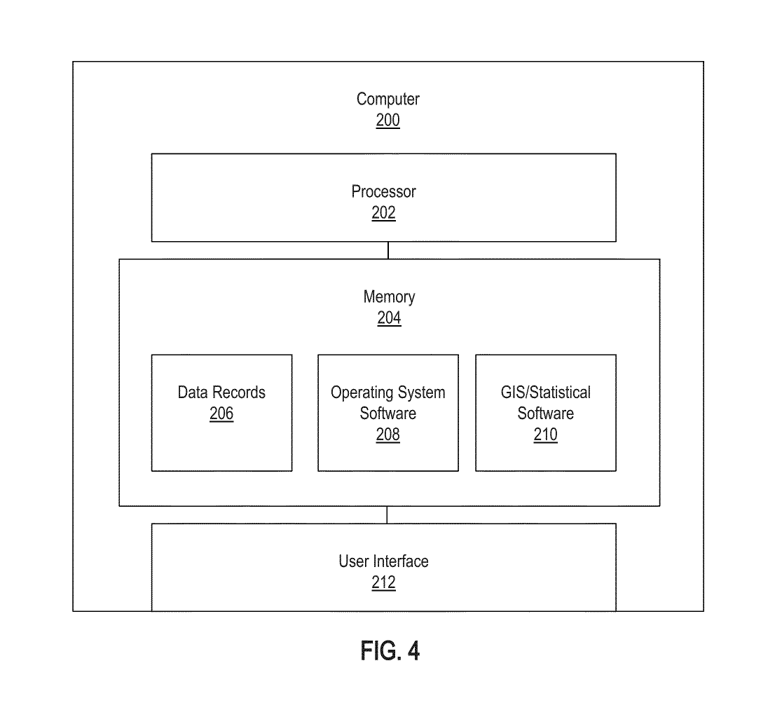

An exemplary system 30 is shown schematically in FIG. 3 for collecting soil data at the field site 10 during the collecting step 112. System 30 may include a communications network 31 and a suitably programmed controller or computer 200, which are discussed further below with reference to FIG. 4.

The illustrative system 30 of FIG. 3 also includes a global positioning system (GPS) receiver 32. In operation, the geographic location (e.g., X, Y, and Z coordinates) of each sampling site 14 may be determined and recorded by locating GPS receiver 32. In this manner, the soil data collected at each sampling site 14 may be associated with the geographic location of that sampling site 14.

The illustrative system 30 of FIG. 3 further includes one or more above-ground sensors 34 and/or a below-ground probe 36 with one or more sensors 38. In this embodiment, sensors 34, 38 may be placed at each sampling site 14 to measure one or more soil parameters. In FIG. 3, after appropriate soil data is collected at a first sampling site 14a and located using GPS receiver 32, the sensors 34, 38 may be moved to collect soil data at a second sampling site 14b, and so on. In another embodiment, the collecting step 112 may involve gathering soil from each sampling site 14 and sending the soil to a lab for analysis.

Certain elements of system 30 may be incorporated into one or more mobile devices or vehicles. Exemplary vehicles include GPS-enabled “Surfer” and “Diver” vehicles provided by C3 Consulting, LLC of Fresno, Calif., as part of the Soil Information System™ (SIS).

Additional information regarding collecting soil data in the collecting step 112 is found in U.S. Pat. No. 6,959,245 to Rooney et al., the disclosure of which is expressly incorporated herein by reference in its entirety.

In step 114 of method 100, a desired and representative number of individual sampling sites 14 may be selected as observation sites 16 for further testing and analysis. For example, if field site 10 is about 40 acres in size, about 15, 20, 25, or more of the sampling sites 14 may be selected as observation sites 16. In embodiments where the number of sampling sites 14 is relatively high, a small percentage of the sampling sites 14 (e.g., 1%, 10%, 20%, or 30% of the sampling sites 14) may be selected as observation sites 16 to make subsequent testing and analysis more manageable. In embodiments where the number of sampling sites 14 is relatively low, most or all of the sampling sites 14 (e.g., 70%, 80%, 90%, or 100% of the sampling sites 14) may be selected as observation sites 16. In other embodiments, about half of the sampling sites 14 (e.g., 40%, 50%, or 60% of the sampling sites 14) may be selected as observation sites 16.

According to an exemplary embodiment of the present disclosure, sampling sites 14 having the most variability in soil data may be identified as observation sites 16. In FIG. 2, three observation sites 16a-16c are shown, where the soil at observation site 16a may have low nutrient levels and the soil at observation site 16c may have high nutrient levels (See Table2 above), and where the soil at observation site 16b may have small root zones (See Table 1 above), for example.

The number and density of observation sites 16 may vary. If the field site 10 of FIG. 2 is 40 acres in size, for example, about 20, 30, 40 or more of the most varied sampling sites 14 may be selected as observation sites 16.

The size of each observation site 16 may also vary. For example, each observation site 16 may have a width that spans about 2, 4, or 6 rows of the test crop and a length of about 10, 20, or 30 feet. In certain embodiments, and as shown in FIG. 2, each observation site 16 may be large enough in size to encompass one or more of the surrounding sampling sites 14. In this case, soil data from a single (e.g., central) sampling site 14 may represent the entire observation site 16, or soil data for the central and surrounding sampling sites 14 may be averaged together to represent the observation site 16.

In step 116 of method 100, the field site 10 may be prepared for planting. The preparing step 116 may involve irrigating the soil to achieve consistent soil moisture levels across the field site 10 of FIG. 2 to support future plant growth. The preparing step 116 may also involve applying minimal amounts of nitrogen-based fertilizers across the field site 10 to support future plant growth.

In step 118 of method 100, a test crop is planted across the field site 10. The type of test crop planted at the field site 10 may vary. For example, the test crop may include a locally adapted corn hybrid. The planting density of the test crop may also vary. For example, the planting density may be about 20,000, 30,000, 40,000 plants/acre or more.

In step 120 of method 100, the test crop is intentionally and uniformly stressed during growth. Stressing the test crop will subject the test crop to less than ideal or normal growing conditions. The stressing step 120 may involve limiting water to the test crop during growth to simulate a drought condition. The stressing step 120 may also involve limiting nutrients to the test crop during growth to simulate a starvation condition. Other stress conditions may be temperature-based, pollution-based, or disease-based, for example. The stressing step 120 may be performed during part of the growing season (e.g., growing stages V6+) or during the entire growing season.

In step 122 of method 100, the field site 10, the test crop, and/or the surrounding environment are monitored. The monitoring step 122 may occur during growth of the test crop. The monitoring step 122 may also occur before and/or after growth of the test crop.

The monitoring step 122 may utilize one or more elements from system 30 of FIG. 3. For example, the monitoring step 122 may involve placing above-ground sensors 34 and/or below-ground sensors 36 at each observation site 16. An exemplary sensor 34, 38 for use during the monitoring step 122 is a moisture sensor which may be placed at each observation site 16 to determine the moisture content of the soil at each observation site 16 during growth of the test crop. The monitoring step 122 may also involve collecting and recording other data, such as historical agronomic practice data, weather data (e.g., temperature, rainfall amount, humidity), planting data (e.g., date), irrigation data (e.g., date, amount), fertilizer, herbicide, and/or insecticide application data (e.g., date, amount), and/or harvesting data (e.g., date), for example.

In step 124 of method 100, crop performance is evaluated at the observation sites 16. The evaluating step 124 may be performed at predetermined time intervals during the growing season and/or at maturity after the growing season. The evaluating step 124 may involve collecting crop performance data, such as plant height, plant yield, total weight, plant weight (e.g., five-plant weight), ear weight, plant flowering, plant biomass, and plant stand, for example, at the observation sites 16 of FIG. 2. Other agronomic performance indices may also be used to evaluate crop performance, such as normalized difference vegetation index (NDVI), anthesis to silking interval (ASI), and the C3 vegetation index (C3VI) used by C3 Consulting, which uses reflectance measurements at certain wavelengths in the visible and near infrared (NIR) range as a proxy for crop biomass. In certain embodiments, crop performance data may be collected by harvesting and measuring (e.g., weighing) the plants.

The evaluating step 124 may also utilize one or more elements from system 30 of FIG. 3. For example, system 30 may include an aerial (e.g., plane or satellite) imaging device 39 to capture images (e.g., multi-spectral, hyper-spectral, visible, and IR images) of the planted crop.

The geographic location of each observation site 16 may be known from the geographic location of the corresponding sampling site(s) 14, such as using GPS receiver 32 of FIG. 3. As discussed above, the soil data collected at each sampling site 14 may be associated with the geographic location of that sampling site 14. Similarly, the crop performance data collected at each observation site 16 may be associated with the geographic location of that observation site 16.

For reasons explained below, the above-described preparing step 116, planting step 118, stressing step 120, monitoring step 122, and evaluating step 124 of method 100 may be referred to herein as “preliminary” steps.

Returning to FIG. 1, in step 126 of method 100, one or more statistical models are developed to correlate the crop performance data from the evaluating step 124 with the soil data from the collecting step 112. The model may be tailored to the particular crop planted during the planting step 118 and the particular stress condition used during the stressing step 120.

The modeling step 126 may involve performing spatial regression analysis to develop an equation for one or more crop performance characteristics as a function of one or more soil parameters. For example, the modeling step 126 may involve performing linear regression analysis to develop a linear best-fit equation for one or more crop performance characteristics as a function of one or more soil parameters. The best-fit equation may be the equation that provides the strongest statistical correlation (e.g., R²) between the crop performance characteristics and the soil parameters. In certain embodiments, individual models may be developed for each desired crop performance characteristic (e.g., a plant height model, a plant yield model). In other embodiments, combined or multivariate models may be developed that take into account a plurality of different performance characteristics.

For simplicity, the model may be based on a desired number of key soil parameters. For example, the model may be based on 2, 3, 4, 5, or more key soil parameters. Key soil parameters may be those having the strongest individual statistical correlation (e.g., R²) with the crop performance data. The remaining, less correlated soil parameters may be eliminated from the model.

Each model may be validated for accuracy using an independent validation dataset. For example, a complete set of soil and crop performance data may be randomly divided into two datasets: one dataset for model development and the other dataset for model validation. Using the validation dataset, a user may ensure that the calculated crop performance values from the model are comparable to the actual crop performance values.

The modeling step 126 may be performed using a computer 200, as shown in FIG. 4. The illustrative computer 200 of FIG. 4 includes a processor 202. Processor 202 may comprise a single processor or include multiple processors, which may be local processors that are located locally within computer 200 or remote processors that are accessible across a network.

The illustrative computer 200 of FIG. 4 also includes a memory 204, which is accessible by processor 202. Memory 204 may be a local memory that is located locally within computer 200 or a remote memory that is accessible across a network. Memory 204 is a computer-readable medium and may be a single storage device or may include multiple storage devices. Computer-readable media may be any available media that may be accessed by processor 202 and includes both volatile and non-volatile media. Further, computer-readable media may be one or both of removable and non-removable media. By way of example, computer-readable media may include, but is not limited to, RAM, ROM, EEPROM, flash memory or other memory technology, CD-ROM, Digital Versatile Disk (DVD) or other optical disk storage, magnetic cassettes, magnetic tape, magnetic disk storage or other magnetic storage devices, or any other medium which may be used to store the desired information and which may be accessed by processor 202.

Memory 204 may include stored data records 206, as shown in FIG. 4. The data records 206 may include the soil data from the collecting step 112 and the crop performance data from the evaluating step 124 of FIG. 1, along with corresponding geographic location data. The data records 206 may also include data from the monitoring step 122 of FIG. 1.

Memory 204 may also include operating system software 208, as shown in FIG. 4. Exemplary operating system software 208 includes, for example, LINUX operating system software, or WINDOWS operating system software available from Microsoft Corporation of Redmond, Wash.

Memory 204 may further include a geographic information system (GIS) software program 210, as shown in FIG. 4. The GIS software program 210 may be capable of statistically analyzing and modeling the geographically-referenced soil data from the collecting step 112 and the crop performance data from the evaluating step 124. If necessary, another statistical software program (not shown) may be provided to interact with the GIS software program 210. The GIS software program 210 may also be capable of managing, calculating, and displaying data based on its geographic location, such as using a map. An exemplary GIS software program 210 is ArcGIS 10.1 available from Environmental Systems Research Institute (ESRI) of Redlands, Calif., and an exemplary statistical software program is JMP available from SAS Institute Inc. of Cary, N.C.

Memory 204 may further include communications software (not shown) to provide access to a communications network, such as network 31 of FIG. 3. In this embodiment, computer 200 may communicate with GPS receiver 32, sensors 34, 38, and imaging device 39 of system 30 via network 31. A suitable communications network includes a local area network, a public switched network, a CAN network, and any type of wired or wireless network. Any exemplary public switched network is the Internet. Exemplary communications software includes e-mail software and internet browser software. Other suitable software which permit computer 200 to communicate with other devices across a network may be used.

The illustrative computer 200 of FIG. 4 further includes a user interface 212 having one or more I/O modules which provide an interface between an operator and computer 200. Exemplary I/O modules include user inputs, such as buttons, switches, keys, a touch display, a keyboard, a mouse, and other suitable devices for providing information to computer 200. Exemplary I/O modules also include user outputs, such as lights, a touch screen display, a printer, a speaker, visual devices, audio devices, tactile devices, and other suitable devices for presenting information to a user.

Returning to method 100 of FIG. 1, the statistical model from the modeling step 126 is applied in step 128 to identify a “zone of uniformity” at the field site. If more than one model is developed during the modeling step 126, the applying step 128 may be performed multiple times to identify a “zone of uniformity” that takes into account some or all of the models from the modeling step 126. The applying step 128 may be performed using the above-described computer 200 of FIG. 4.

The applying step 128 may involve inputting the soil data from the collecting step 112 into the model and using the model to calculate a predicted crop performance value at each location. In the illustrated embodiment of FIG. 2, for example, the applying step 128 may involve inputting the soil data collected from each sampling site 14 into the model and using the model to calculate a predicted crop performance value for each sampling site 14.

The “zone of uniformity” represents an area of the field site where the predicted crop performance values from the model are uniform within an acceptable tolerance. In the illustrated embodiment of FIG. 2, for example, the “zone of uniformity” 18 (shown with phantom borders) represents an area of the field site 10 (shown with solid borders) where the predicted crop performance values from the model are uniform within an acceptable tolerance. The acceptable tolerance may vary depending on the crop performance parameter, the range of crop performance values, and other factors. For example, the acceptable tolerance may be as low as about +/−0.5%, 1%, or 2% and as high as about +/−3%, 4%, or 5%.

The “zone of uniformity” may be defined by the geographic coordinates of each corner or border, for example, or by another suitable method. The size and shape of the “zone of uniformity” 18 may vary. Although the illustrative “zone of uniformity” 18 of FIG. 2 is irregular in shape, the “zone of uniformity” 18 may also be circular, triangular, or rectangular in shape, for example. Also, although the illustrative “zone of uniformity” 18 is a single continuous area in FIG. 2, the “zone of uniformity” 18 may also include multiple distinct or spaced-apart areas.

According to an exemplary embodiment of the present disclosure, the applying step 128 may be performed by arranging the predicted crop performance values from the model from low to high on a numbered scale (e.g., 0 to 10, 0 to 100). In this embodiment, crop performance values that share the same number on the scale may be located within an acceptable tolerance. A user may identify the “zone of uniformity” as an area where the predicted crop performance values share the same number on the scale. In this embodiment, the size of the scale may be selected to achieve a desired tolerance. If the acceptable tolerance at each level of the scale is relatively small or tight, the predicted crop performance values may be arranged on a relatively large scale (e.g., 0 to 100). If the acceptable tolerance at each level of the scale is relatively large, the predicted crop performance values may be arranged on a relatively small scale (e.g., 0 to 10).

According to another exemplary embodiment of the present disclosure, the applying step 128 may be performed visually using a uniformity map. In this embodiment, different crop performance values or ranges of crop performance values from the model may be associated with different colors or symbols. A user may identify the “zone of uniformity” as an area having a substantially uniform or homogenous color.

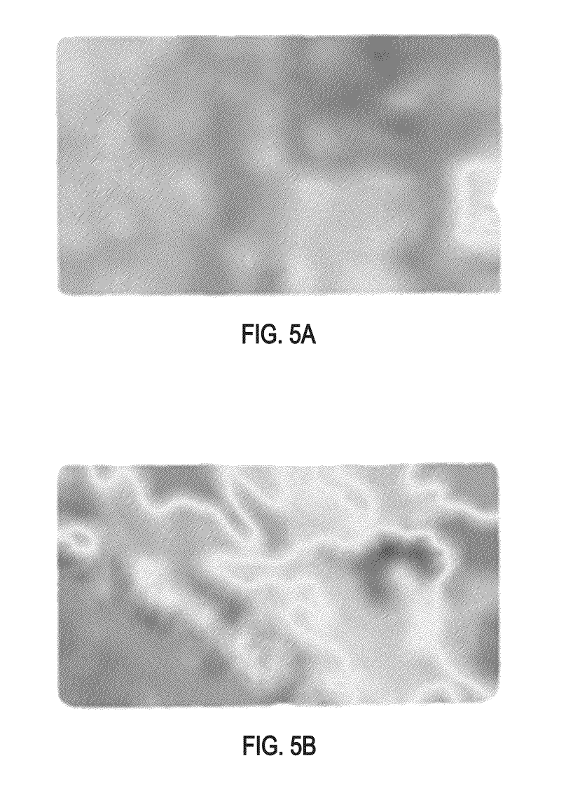

For example, the user may identify the substantially uniform area shown in FIG. 5A as the “zone of uniformity,” rather than the more variable area shown in FIG. 5B. In embodiments where the predicted crop performance values are arranged on a numbered scale, as discussed above, different colors may be assigned to each number on the scale to facilitate selection of the “zone of uniformity.”

Returning to FIG. 1, a subsequent preparing step 130, a subsequent planting step 132, a subsequent stressing step 134, a subsequent monitoring step 136, and a subsequent evaluating step 138 may be performed in the “zone of uniformity” identified during the applying step 128. The subsequent steps 130-138 may be generally similar to the corresponding preliminary steps 116-124 described above. However, with reference to FIG. 2, the preliminary steps 116-124 were performed across the field site 10, whereas the subsequent steps 130-138 may be limited to the “zone of uniformity” 18. According to the model(s) from the modeling step 126, the soil located in the “zone of uniformity” 18 should have little or no variability in predetermined soil parameters that will significantly impact crop performance during the subsequent planting step 132 and stressing step 134. In other words, planting the crops in the “zone of uniformity” 18 may reduce or eliminate exposure to predetermined soil parameters that would significantly impact crop performance during the subsequent planting step 132 and stressing step 134. Thus, performing the subsequent planting step 132 and stressing step 134 in the “zone of uniformity” 18 may enhance the probability of a successful agronomic stress trial to generate accurate and reliable phenotyping.

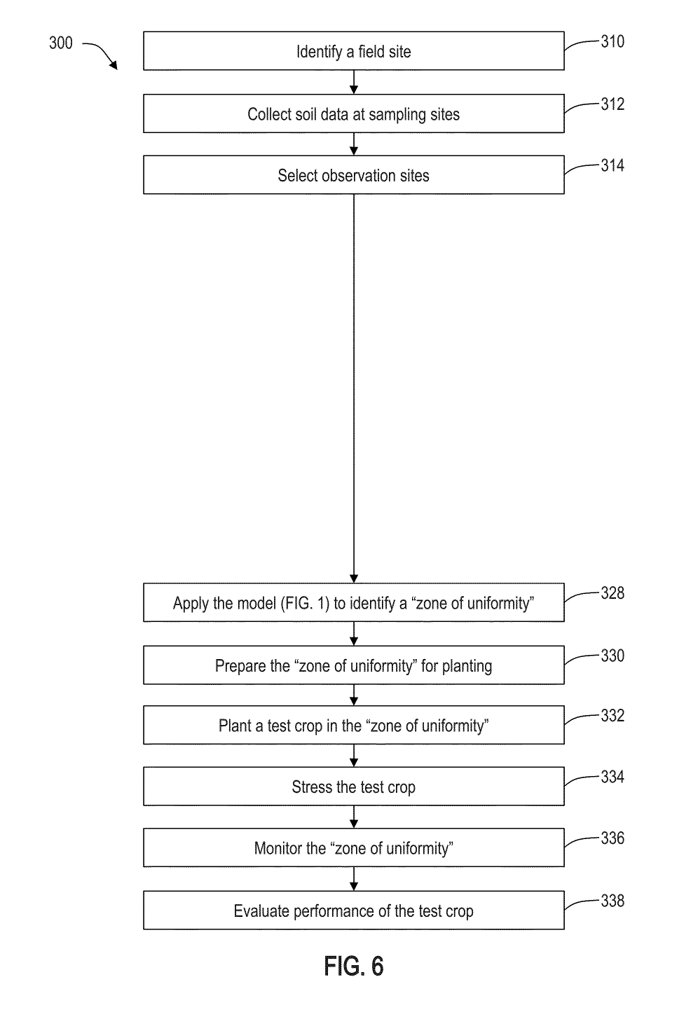

Referring next to FIG. 6, another method 300 is provided for characterizing a future field site and selecting a “zone of uniformity” at the field site for agronomic stress trial. Method 300 of FIG. 6 may rely on the above-described model(s) from method 100 of FIG. 1 to identify future “zones of uniformity” to stress test the same crop from FIG. 1 or a next-generation crop. Advantageously, unlike method 100 of FIG. 1, method 300 of FIG. 6 may not require a preliminary preparing step, a preliminary planting step, a preliminary stressing step, a preliminary monitoring step, a preliminary evaluating step, or a modeling step, for example. Thus, by relying on the above-described model(s) from method 100 of FIG. 1, future “zones of uniformity” may be identified quickly, efficiently, and accurately, even for future field sites that are remote from the initial field site that was used to develop the model.

As shown in FIG. 6, method 300 may include an identifying step 310 (which is similar to the identifying step 110 of method 100), a soil data collecting step 312 (which is similar to the collecting step 112 of method 100), and an identifying step 314 (which is similar to the identifying step 114 of method 100). For improved efficiency, the collecting step 312 may be limited to the key soil parameters included in the model(s), rather than a complete survey of soil parameters. Based on the soil data collected during the collecting step 312, the above-described model(s) from method 100 may be applied in step 328 (which is similar to the applying step 128 of method 100) to identify a “zone of uniformity” at the field site. This “zone of uniformity” may be used to perform a preparing step 330 (which is similar to the subsequent preparing step 130 of method 100), a planting step 332 (which is similar to the subsequent planting step 132 of method 100), a stressing step 334 (which is similar to the subsequent stressing step 134 of method 100), a monitoring step 336 (which is similar to the subsequent monitoring step 136 of method 100), and an evaluating step 338 (which is similar to the subsequent evaluating step 138 of method 100). Performing the planting step 332 and the stressing step 334 in the “zone of uniformity” may enhance the probability of a successful agronomic stress trial to generate accurate and reliable phenotyping.

### EXAMPLE

Two fields (Dixon and Yolo) were identified in the Woodland, Calif. area. Each field was approximately 40 acres in size. The soil data set forth in Table 1 and Table 2 above was collected. GPS data was used to associate the collected soil data with its geographic location.

The fields were planted with 2V707 corn hybrid seeds supplied by Mycogen Seeds of Minneapolis, Minn., at a density of about 34,000 to 36,000 plants/acre. Standard agronomic practices typical of the area were used except for creating (1) a moderate nitrogen deficit condition and (2) a water deficit condition. To create the moderate nitrogen stress condition, the total amount of nitrogen-based fertilizer applied to the fields was limited to 100 pounds nitrogen/acre. To create the water stress condition, irrigation was provided in a sufficient amount immediately after planting and during the early growing stages, but irrigation was withheld starting at the V6-V8 growing stages and for the remainder of the growing season to limit plant water use (no more than 250-300 mm of water for the growing season). “Rescue” irrigations were only applied if severe signs of stress were consistently noticed.

The following observations were collected and recorded during the growing season: weather data; soil physical characteristics; soil moisture content; field routine scouting; agronomic practices including crop history over 2 years; date and rates for all application of fertilizer, herbicide, or insecticide; date and amount for each irrigation event; and planting and harvesting dates.

In each field, 20 observation sites were identified for performance evaluation. Each observation site had an area of 4-rows by 20 feet. The following performance data was collected at each observation site: total weight; ear weight; five-plant weight; plant height at growing stage V11; and ASI. Also, the C3VI performance values at each observation site were determined using aerial imagery.

For each performance value to be modeled, key physical and chemical soil parameters were identified using forward step-wise regression analysis. For the C3VI performance value, for example, the key physical soil parameters identified in Table 3 and the key chemical soil parameters identified in Table 4 were found to have the highest correlation coefficients. The sub-surface nitrate-N content (C24) was also included as a key chemical soil parameter in Table 4 based on experience. These key soil parameters were selected for modeling. The numbers in Table 3 and Table 4 correspond to the numbers in Table 1 and Table 2, respectively.

TABLE 3
Key Physical Soil Parameters for C3VI

| | | Correlation Coefficient | |
|---|---|---|---|
| Number | Parameter | (R) | (R²) |
| P8 | Root Zone Permanent Wilting Point | −0.74 | 0.55 |
| P3 | Sub-surface Clay | −0.73 | 0.54 |
| P7 | Root Zone Field Capacity | −0.71 | 0.51 |
| P2 | Surface Clay | −0.71 | 0.50 |
| P5 | Drainage Potential | 0.70 | 0.49 |
| P13 | Sub-surface Sand | 0.65 | 0.42 |
| P10 | Root Zone Saturated Hydraulic | 0.64 | 0.41 |
| | Conductivity | | |
| P11 | Root Zone Saturation | −0.63 | 0.39 |
| P12 | Surface Sand | 0.58 | 0.34 |
| P9 | Root Zone Plant Available Water | −0.54 | 0.29 |

TABLE 4
Key Chemical Soil Parameters for C3VI

| | | Correlation Coefficient | |
|---|---|---|---|
| Number | Parameter | (R) | (R²) |
| C9 | Surface Calcium Magnesium Ratio | 0.85 | 0.72 |
| C19 | Surface Magnesium Base Saturation | −0.85 | 0.72 |
| C17 | Surface Magnesium | −0.84 | 0.70 |
| C7 | Surface Calcium Base Saturation | 0.83 | 0.69 |
| C25 | Nutrient Holding Capacity | −0.82 | 0.68 |
| C29 | Sub-surface pH | −0.82 | 0.67 |
| C33 | Sub-surface Phosphorus Availability | 0.82 | 0.67 |
| C11 | Surface Cation Exchange Capacity | −0.81 | 0.66 |
| C26 | Surface Organic Matter | −0.80 | 0.64 |
| C4 | Sub-surface Boron | −0.78 | 0.61 |
| C24 | Sub-surface Nitrate-N | N/A | N/A |

Soil and performance data from the Dixon and Yolo fields were merged together and then randomly divided into two datasets: one dataset for model development and the other dataset for model validation. The following multiple linear regression models (1)-(6) were developed using the development dataset and validated using the validation dataset.

Total Weight=44.0+2.2(C7)+2.2(C19)+0.6(C24)−30.0(C29)

R²=0.72 (1)

Ear Weight=23,368.7+845.2(P7)−22.5(C23)−4,529.2(C26)−7,303.4(C27)

R²=0.60 (2)

Five-Plant Weight=45,773.9−107.2(C7)−54.0(C19)−5,321.5(C29)

+34.2(C23)+262.9(P11)

R²=0.72 (3)

Plant Height(V11)=1,134.1+47.3(C4)−1.5(C12)+0.9(C19)

−4.8(C25)−89.6(C29)

R²=0.80 (4)

ASI=107.5−0.3(P12)−4.8(P11)−1.4(C4)+7.0(C27)

R²=0.57 (5)

C3VI=349.6+1.6(P2)−9.3(P11)+1.6(C7)−1.1(C25)+1.3(C24)

R²=0.90 (5)

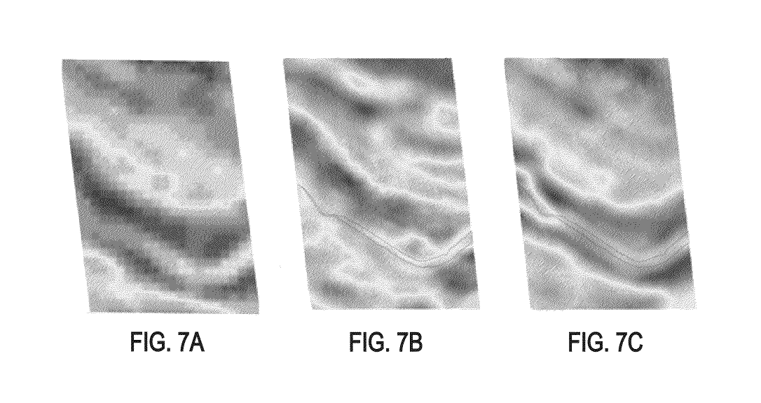

The models were applied to the Dixon and Yolo fields to perform uniformity mapping. The application of model (6) for C3VI at the Dixon field is shown in FIG. 7A. The application of model (1) for total weight at the Dixon field is shown in FIG. 7B. The application of model (4) for plant height at the Dixon field is shown in FIG. 7C. Although different models were used to generate the uniformity maps of FIGS. 7A-7C, similarities are evident between the uniformity maps.

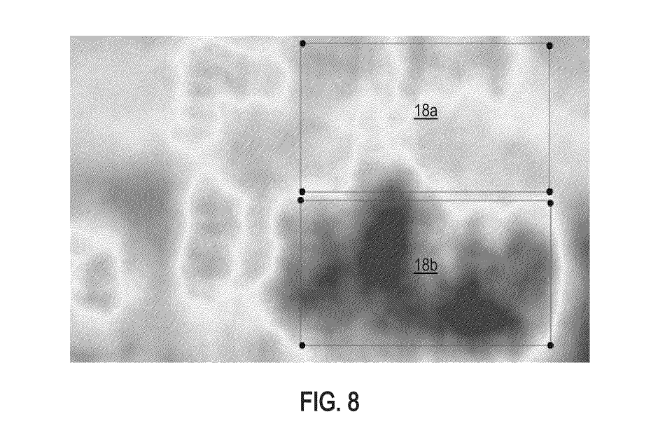

One or more areas of uniform color representing statistically significant soil uniformity were then identified as “zones of uniformity.” Two potential “zones of uniformity” 18a and 18b are shown in FIG. 8.

The models were then applied to fields other than the Dixon and Yolo fields in the Woodland, Calif. area to identify “zones of uniformity” in the other fields for agronomic testing. A potential “zone of uniformity” is shown in FIG. 5A, in contrast to a more variable area shown in FIG. 5B.

While this invention has been described as relative to exemplary designs, the present invention may be further modified within the spirit and scope of this disclosure. Further, this application is intended to cover such departures from the present disclosure as come within known or customary practice in the art to which this invention pertains.

1. A method for performing an agronomic test at a field site, the method comprising:
identifying a zone of the field site having minimal variation in at least one predetermined soil parameter, the at least one predetermined soil parameter affecting agronomic performance during the test;
planting a crop in the zone of the field site; and
subjecting the planted crop to the test.

2. The method of claim 1, further comprising predicting a performance value of the crop based on the at least one predetermined soil parameter.

3. The method of claim 2, wherein the predicting step occurs before the planting step and the subjecting step.

4. The method of claim 1, further comprising collecting soil data regarding the field site.

5. The method of claim 5, wherein the collecting step occurs before the planting step and the subjecting step.

6. The method of claim 1, wherein the test comprises at least one of a nutrient deficit test and a water deficit test.

7. The method of claim 1, wherein the identifying step comprises applying a model of agronomic performance as a function of the at least one predetermined soil parameter.

8. The method of claim 1, wherein the at least one predetermined soil parameter comprises one of root zone permanent wilting point, sub-surface clay content, root zone field capacity, surface clay content, drainage potential, sub-surface sand content, root zone saturated hydraulic conductivity, root zone saturation, surface sand content, and root zone plant available water.

9. The method of claim 1, wherein the at least one predetermined soil parameter comprises one of surface calcium magnesium ratio, surface magnesium base saturation, surface magnesium content, surface calcium base saturation, nutrient holding capacity, sub-surface pH, sub-surface phosphorus availability, surface cation exchange capacity, surface organic matter, sub-surface boron, and sub-surface nitrate content.

10. The method of claim 1, wherein the at least one predetermined soil parameter comprises one of surface clay content, root zone saturation, surface calcium base saturation, nutrient holding capacity, and sub-surface nitrate content.

11. A method for selecting a field site for an agronomic test, the method comprising:
planting a test crop;
subjecting the planted test crop to the test;
determining at least one soil parameter that affects agronomic performance of the test crop during the test; and
selecting a zone of the field site having minimal variation in the at least one soil parameter.

12. The method of claim 11, wherein the subjecting step comprises subjecting the planted test crop to a stress test.

13. The method of claim 11, wherein the subjecting step comprises subjecting the planted test crop to at least one of a nutrient deficit condition and a water deficit condition.

14. The method of claim 11, wherein the determining step comprises developing a model of agronomic performance as a function of the at least one soil parameter.

15. The method of claim 14, wherein the model comprises a best-fit linear equation of agronomic performance as a function of the at least one soil parameter.

16. The method of claim 11, further comprising:
planting a second test crop in the zone; and
subjecting the second planted test crop to the test.

17. The method of claim 16, further comprising:
identifying another field site remote from the field site of claim 11;
selecting a third zone of the other field site having minimal variation in the at least one soil parameter;
planting a third test crop in the third zone; and
subjecting the third planted test crop to the test.

18. A method for selecting a field site for an agronomic test, the method comprising:
planting a first test crop;
subjecting the first planted test crop to the test;
determining at least one soil parameter that affects agronomic performance of the first test crop during the test;
selecting a zone of the field site having minimal variation in the at least one soil parameter;
planting a second test crop in the zone; and
subjecting the second planted test crop to the test.

19. The method of claim 18, wherein second test crop is planted remotely from the first test crop.

20. The method of claim 18, wherein the determining step comprises developing a best-fit equation of agronomic performance as a function of the at least one soil parameter.
